(19) United States
(12) Patent Application Publication
(10) Pub. No.: US 2022/0202058 A1
(43) Pub. Date: Jun. 30, 2022

(54) METHOD FOR CONTROLLING FOOD PRINTER

(71) Applicant: Panasonic Intellectual Property Management Co., Ltd., Osaka (JP)

(72) Inventors: HIROSHI YAHATA, Osaka (JP); TAKAHIRO NISHI, Nara (JP); TADAMASA TOMA, Osaka (JP); TOSHIYASU SUGIO, Osaka (JP); CHRISTOPHER JOHN WRIGHT, London (GB); BERNADETTE ELLIOTT BOWMAN, London (GB); DAVID MICHAEL DUFFY, Zurich (CH)

(21) Appl. No.: 17/695,380

(22) Filed: Mar. 15, 2022

Related U.S. Application Data
(63) Continuation of application No. PCT/JP2020/047892, filed on Dec. 22, 2020.

(30) Foreign Application Priority Data
Apr. 6, 2020 (JP) 2020-068603

(51) Int. Cl. A23P 30/00; A23P 20/25

(52) U.S. Cl. CPC A23P 30/00; A23P 2020/253; A23P 20/25

(57) ABSTRACT

A control method includes: acquiring chewing/swallowing information via a network from a sensing device with which a user is equipped, wherein the chewing/swallowing information is related to chewing of the user when the user eats a first printed food; determining based on the chewing/swallowing information, a swallow cycle duration of the user, and determining based on a first hardness and the swallow cycle duration, a second hardness for a second printed food to be created by a food printer; and transmitting print control information to the food printer via the network, wherein the print control information being used for causing the food printer to create the second printed food having the determined second hardness.

### BACKGROUND

### 1. Technical Field

The present disclosure relates to a method for controlling a food printer.

### 2. Description of the Related Art

Japanese Unexamined Patent Application Publication No. 2014-054269 discloses an oral function training implement that makes it possible to recover, maintain, or improve oral function, and allows training to be performed in a manner similar to the actual swallowing motion. Specifically, the oral function training implement disclosed in Japanese Unexamined Patent Application Publication No. 2014-054269 includes a grip, and an insertion unit designed for insertion into the oral cavity. The insertion unit is provided with a flexible elastic body with a hollow area defined therein. The elastic body includes a hole, and a slit that communicates the hollow area with the outside.

International Publication No. 2014/190168 discloses a 3D printer used for food manufacture.

### SUMMARY

One non-limiting and exemplary embodiment provides further improvements over the techniques described in Japanese Unexamined Patent Application Publication No. 2014-054269 and International Publication No. 2014/190168.

In one general aspect, the techniques disclosed here feature a method for controlling a food printer in a food-material providing system. The food printer is used to create a first printed food having a first hardness by using a material in paste form. The method includes: acquiring chewing/swallowing information via a network from a sensing device with which a user is equipped, wherein the chewing/swallowing information is related to chewing of the user when the user eats the first printed food; determining based on the chewing/swallowing information, a swallow cycle duration of the user, and determining based on the first hardness and the swallow cycle duration, a second hardness for a second printed food to be created by the food printer; and transmitting print control information to the food printer via the network, wherein the print control information is used for causing the food printer to create the second printed food having the determined second hardness.

Additional benefits and advantages of the disclosed embodiments will become apparent from the specification and drawings. The benefits and/or advantages may be individually obtained by the various embodiments and features of the specification and drawings, which need not all be provided in order to obtain one or more of such benefits and/or advantages.

### BRIEF DESCRIPTION OF THE DRAWINGS

FIG. 1 is a block diagram illustrating an exemplary general configuration of an information system according to an embodiment of the present disclosure;

FIG. 2 illustrates an exemplary data structure of a chewing/swallowing information database;

FIG. 3 is a sequence diagram illustrating an overview of processing performed by the information system illustrated in FIG. 1;

FIG. 4 is a flowchart according to the embodiment, providing a detailed illustration of processing performed by a server; and

FIG. 5 illustrates the progression of mean swallow cycle duration over time.

### DETAILED DESCRIPTIONS

### Underlying Knowledge Forming Basis of the Present Disclosure

Chewing function and swallowing function (to be referred to as “chewing and swallowing function” hereinafter) are known to decrease with aging. Severe impairment of chewing and swallowing function may have consequences such as deteriorated nutritional status resulting from the inability or difficulty to eat and drink, decreased quality of life (QOL) resulting from the loss of the pleasure of eating, and development of aspiration pneumonia resulting from entry of food or drink into the airway. Aspiration pneumonia, in particular, is among the leading causes of death for the elderly. Accordingly, it is becoming an urgent issue to improve the chewing and swallowing function of the elderly.

If a soft food is provided to an elderly person with decreased chewing and swallowing function for the reason that such a food is easy to eat, this may temporarily allow the elderly person to smoothly ingest the food. However, continuing to provide such a food to the elderly person may further exacerbate the deterioration of the chewing and swallowing function of the elderly person.

Conversely, if a food that requires much chewing is given to an elderly person, it takes a greater number of chews, and a longer swallow cycle duration for the elderly person to eat the food. This may make it temporarily impossible or difficult for the elderly person to smoothly ingest the food. However, continuing to provide such a food to the elderly person can potentially improve the chewing and swallowing function of the elderly person. This leads to reduced number of chews for the same food, which results in reduced swallow cycle duration.

According to Japanese Unexamined Patent Application Publication No. 2014-054269 mentioned above, the training implement is inserted into the user's oral cavity, and training is performed in a manner similar to the actual swallowing motion. The technique according to Japanese Unexamined Patent Application Publication No. 2014-054269, however, merely involves making the user perform a swallowing motion in a simulated fashion, and does not involve making the user actually chew a real food and perform the actual swallowing motion.

International Publication No. 2014/190168 neither describes nor suggests using food manufactured by a 3D printer to improve the chewing and swallowing function of the elderly.

The above-mentioned knowledge has led the present inventors to discover a method for controlling a food printer that makes it possible to improve the chewing and swallowing function of the user through provision of a food with a suitable hardness.

According to an aspect of the present disclosure, there is provided a method for controlling a food printer in a food-material providing system. The food printer is used to create a first printed food having a first hardness by using a material in paste form. The method includes: acquiring chewing/swallowing information via a network from a sensing device with which a user is equipped, wherein the chewing/swallowing information is related to chewing of the user when the user eats the first printed food; determining based on the chewing/swallowing information, a swallow cycle duration of the user, and determining based on the first hardness and the swallow cycle duration, a second hardness for a second printed food to be created by the food printer; and transmitting print control information to the food printer via the network, wherein the print control information is used for causing the food printer to create the second printed food having the determined second hardness.

According to the above-mentioned configuration, the chewing/swallowing information related to chewing of the user when the user eats the first printed food having the first hardness is acquired from the sensing device via the network. The swallow cycle duration of the user is determined based on the chewing/swallowing information. The second hardness is determined based on the determined swallow cycle duration and the first hardness. The print control information for causing the food printer to create the second printed food having the determined second hardness is transmitted to the food printer via the network.

Consequently, based on the swallow cycle duration when the user eats the first printed food having the first hardness, a suitable second hardness for improving the chewing and swallowing function of the user can be determined. This makes it possible to make the food printer create the second printed food having the determined second hardness, and have the created second printed food eaten by the user. This makes it possible to improve the chewing and swallowing function of the user.

In the method mentioned above, the swallow cycle duration may correspond a period of time from when the user starts chewing a bite of the first printed food to when the user swallows the bite of the first printed food.

The above-mentioned configuration makes it possible to clearly define the start timing and end timing of the swallow cycle duration.

In the method mentioned above, the print control information may include a print condition for, if the swallow cycle duration of the user is less than a predetermined cycle duration, creating the second printed food having the second hardness greater than the first hardness.

A longer swallow cycle duration for a given food may be associated with decreased chewing and swallowing function of the user. If only soft food materials are continuously provided to the user with decreased chewing and swallowing function, the chewing and swallowing function of the user does not improve. According to the above-mentioned configuration, if the swallow cycle duration is less than a predetermined cycle duration, a second hardness greater than the first hardness is determined as the hardness of the food to be provided. This results in increased number of chews taken to swallow the food, which makes it possible to improve the chewing and swallowing function of the user.

In the method mentioned above, the sensing device may include an acceleration sensor, and the chewing/swallowing information may include acceleration information, the acceleration information representing an acceleration detected by the acceleration sensor.

According to the above-mentioned configuration, the swallow cycle duration is determined based on the acceleration information detected by the acceleration sensor. This makes it possible to accurately determine the swallow cycle duration.

In the method mentioned above, the acceleration sensor may be installed on one of a chopstick, a fork, or a spoon of the user, and a beginning of the swallow cycle duration may be determined by using one of a first timing determined based on the acceleration information or a second timing determined based on the acceleration information. The first timing represents a timing when the user raises the one of a chopstick, a fork, or a spoon, and the second timing represents a timing when the user lowers the one of a chopstick, a fork, or a spoon.

According to the above-mentioned configuration, based on acceleration information detected by the acceleration sensor installed on the user's chopstick, fork, or spoon, the first timing when the user raises the chopstick, the fork, or the spoon, or the second timing when the user lowers the chopstick, the fork, or the spoon is detected. The beginning of the swallow cycle duration is detected by using the first timing or the second timing. This makes it possible to detect the beginning of the swallow cycle duration in everyday life of the user.

In the method mentioned above, the sensing device may detect an electromyographic potential, and an end of the swallow cycle duration may be determined based on the detected electromyographic potential.

According to the above-mentioned configuration, a sensor that detects an electromyographic potential is used to detect the user's electromyographic potential, and the end of the swallow cycle duration is determined based on the electromyographic potential. This makes it possible to accurately determine the end of the swallow cycle duration.

In the method mentioned above, the sensing device may be installed on eyeglasses of the user.

According to the above-mentioned configuration, the user simply puts on eyeglasses to allow detection of the user's electromyographic potential, and the swallow cycle duration of the user is determined from the detected electromyographic potential. This makes it possible to determine the swallow cycle duration in everyday life of the user.

In the method mentioned above, the sensing device may detect chewing sound, and an end of the swallow cycle duration may be determined based on the detected chewing sound.

According to the above-mentioned configuration, the swallow cycle duration is determined based on the chewing sound. This makes it possible to accurately determine the swallow cycle duration.

In the method mentioned above, the sensing device may include a microphone installed on a necklace of the user.

According to the above-mentioned configuration, a device that detects chewing sound is installed on a necklace of the user. This makes it possible to determine the end of the swallow cycle duration in everyday life of the user.

In the method mentioned above, the sensing device may include an earphone-type microphone of the user.

According to the above-mentioned configuration, the user simply puts on an earphone-type microphone to allow determination of the end of the swallow cycle duration.

In the method mentioned above, the second printed food may include a three-dimensional structure including a number of holes, and the second hardness may be adjusted by increasing or decreasing the number of holes.

According to the above-mentioned configuration, the second hardness of the second printed food can be changed through a process that is simple for the food printer, such as increasing or decreasing the number of holes in the second printed food.

In the method mentioned above, the print control information may specify the number of holes per unit volume.

According to the above-mentioned configuration, the print control information specifies the number of holes per unit volume of the second printed food. This allows for creation of the second printed food without non-uniformity of hardness.

In the method mentioned above, the second printed food may comprise a three-dimensional structure including a plurality of layers, the plurality of layers including a first layer with a third hardness and a second layer with a fourth hardness, and the print control information may include a print condition for causing the third hardness to be greater than the fourth hardness.

According to the above-mentioned configuration, the second printed food includes plural layers including a first layer with a third hardness and a second layer with a fourth hardness, and the third hardness is made greater than the fourth hardness. Consequently, for example, a second printed food with a hard surface (first layer) and a soft interior (second layer) can be created. This makes it possible to create a second printed food having a texture such that as the user crushes its hard surface with the teeth, its contents with taste mix with saliva and melt out from the inside. This induces saliva production, which helps to efficiently improve the chewing and swallowing function of the user.

In the method mentioned above, the print control information may specify a temperature at which to bake the second printed food.

According to the above-mentioned configuration, the print control information includes information specifying the temperature at which to bake the second printed food. Accordingly, for example, the hardness of the second printed food can be adjusted by controlling or specifying at what temperature each individual portion of the second printed food is to heated with a laser output unit in creating the second printed food, or by controlling or specifying at what temperature the entire second printed food is to be heated with another food preparation appliance (e.g., an oven) after the second printed food is created.

According to another aspect of the present disclosure, there is provided a method for controlling a food printer of a food-material providing system. The food printer is a used to create a first printed food having a first hardness by using a material in paste form. The method includes: acquiring chewing/swallowing information via a network from a sensing device with which a user is equipped, wherein the chewing/swallowing information representing a swallow cycle duration of the user when the user eats the first printed food; determining based on the first hardness and the chewing/swallowing information, a second hardness for a second printed food to be created by the food printer; and transmitting print control information to the food printer via the network, wherein the print control information is used for causing the food printer to create the second printed food having the determined second hardness.

According to the above-mentioned configuration, chewing/swallowing information, which is information representing the swallow cycle duration of the user when the user eats the first printed food having the first hardness, is acquired from the sensing device via the network. The second hardness is determined based on the first hardness and the chewing/swallowing information. The print control information for causing the food printer to create the second printed food having the determined second hardness is transmitted to the food printer via the network.

Consequently, based on the swallow cycle duration when the user eats the first printed food having the first hardness, a suitable second hardness for improving the chewing and swallowing function of the user can be determined. This makes it possible to make the food printer create the second printed food having the determined second hardness, and have the created second printed food eaten by the user. This makes it possible to improve the chewing and swallowing function of the user. This configuration is useful if the sensing device used is capable of detecting the swallow cycle duration.

In the method mentioned above, the swallow cycle duration may include a period of time from when the user starts chewing a bite of the first printed food to when the user swallows the bite of the first printed food.

The above-mentioned configuration makes it possible to clearly define the start timing and end timing of the swallow cycle duration.

In the method mentioned above, the print control information may include a print condition for, if the swallow cycle duration of the user is less than a predetermined cycle duration, creating the second printed food having the second hardness greater than the first hardness.

A longer swallow cycle duration for a given food may be associated with decreased chewing and swallowing function of the user. If only soft food materials are continuously provided to the user with decreased chewing and swallowing function, the chewing and swallowing function of the user does not improve. According to the above-mentioned configuration, if the swallow cycle duration is less than a predetermined cycle duration, a second hardness greater than the first hardness is determined as the hardness of the food to be provided. This results in increased number of chews taken to swallow the food, which makes it possible to improve the chewing and swallowing function of the user.

In the method mentioned above, the sensing device may include a camera, and a beginning and an end of the swallow cycle duration of the user may be determined based on a result of image recognition performed by using an image obtained with the camera.

Since the sensing device is implemented as a camera, by applying an image recognition process to an image obtained with the camera, the beginning and end of the swallow cycle duration can be determined.

The present disclosure can be implemented also as a program for causing a computer to execute various characteristic features included in the control method mentioned above, or as a food-material providing system that operates in accordance with the program. It is needless to mention that such a computer program can be distributed via a computer-readable non-transitory recording medium such as a CD-ROM, or via a communications network such as the Internet.

Embodiments described below each represent one specific implementation of the present disclosure. Specific details set forth in the following description of embodiments, such as numeric values, shapes, components, steps, and the order of steps, are for illustrative purposes only and not intended to limit the scope of the present disclosure. Those components in the following description of embodiments which are not cited in the independent claim representing the most generic concept of the present disclosure will be described as optional components. For all embodiments of the present disclosure below, the features of individual embodiments may be used in combination.

### EMBODIMENTS

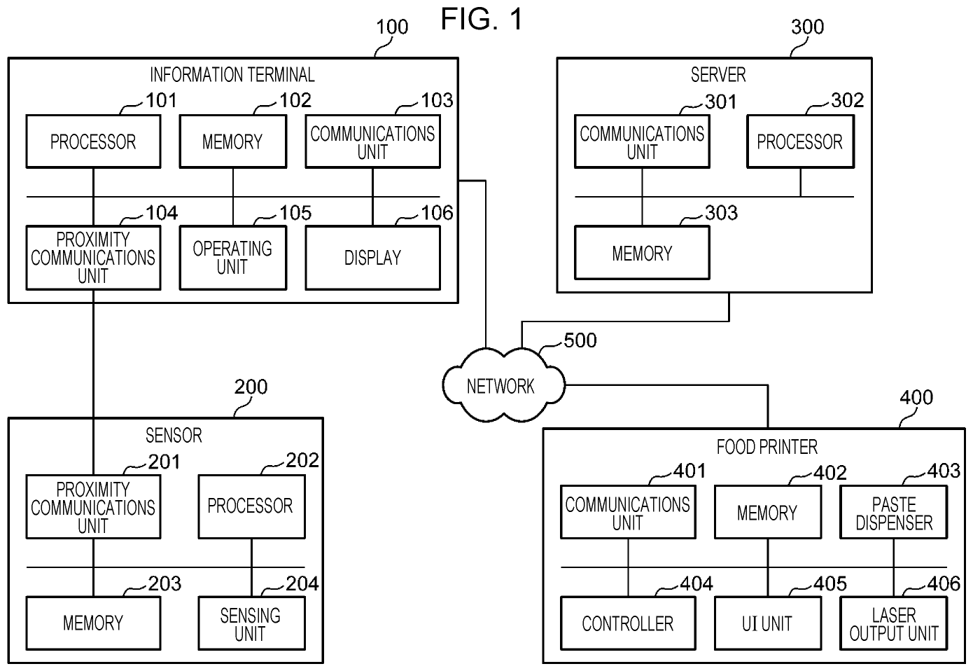

FIG. 1 is a block diagram illustrating an exemplary general configuration of an information system according to an embodiment of the present disclosure. The information system includes an information terminal 100, a sensor 200, a server 300, and a food printer 400. The server 300 and the food printer 400 each represent an example of a food-material providing system. The information terminal 100, the server 300, and the food printer 400 are capable of communicating with each other via a network 500. The information terminal 100 and the sensor 200 are capable of communicating with each other through proximity wireless communication. The network 500 is implemented as, for example, a wide area network including an Internet communications network and a mobile phone communications network. For proximity wireless communication, for example, a wireless technology such as Bluetooth (registered trademark) or NFC is used.

The information terminal 100 is implemented as, for example, a mobile information processing apparatus such as a smartphone or a tablet terminal. However, this is intended to be illustrative only. Alternatively, the information terminal 100 may be implemented as a desktop information processing apparatus.

The information terminal 100 is carried by a user who receives a food-material providing service provided by the food-material providing system. The information terminal 100 includes a processor 101, a memory 102, a communications unit 103, a proximity communications unit 104, an operating unit 105, and a display 106.

The processor 101 is implemented as, for example, a CPU. The processor 101 is responsible for overall control of the information terminal 100. The processor 101 executes the operating system of the information terminal 100, and executes a sensing application for receiving sensing data from the sensor 200 and transmitting the sensing data to the server 300.

The memory 102 is implemented as, for example, a rewritable non-volatile storage device such as a flash memory. The memory 102 stores, for example, the operating system and the sensing application. The communications unit 103 is implemented as a communications circuit for connecting the information terminal 100 to the network 500. The communications unit 103 transmits sensing data to the server 300 via the network 500. The sensing data in this case is sensing data transmitted from the sensor 200 via proximity wireless communication and received by the proximity communications unit 104. The proximity communications unit 104 is implemented as a communications circuit that complies with a proximity wireless communications standard. The proximity communications unit 104 receives sensing data transmitted from the sensor 200.

The operating unit 105 is implemented as an input device such as a touchscreen if the information terminal 100 is implemented as a mobile information processing apparatus. The operating unit 105 is implemented as an input device such as a keyboard and a mouse if the information terminal 100 is implemented as a desktop information processing apparatus. The display 106 is implemented as a display device such as an organic EL display or a liquid crystal display.

The sensor 200 is implemented as a sensing device installed on the user. The sensor 200 includes a proximity communications unit 201, a processor 202, a memory 203, and a sensing unit 204. The proximity communications unit 201 is implemented as a communications circuit that complies with a proximity wireless communications standard. The proximity communications unit 201 transmits sensing data detected by the sensing unit 204 to the information terminal 100.

The processor 202 is implemented as, for example, a CPU, and is responsible for overall control of the sensor 200. The memory 203 is implemented as, for example, a non-volatile rewritable storage device such as a flash memory. The memory 203 temporarily stores, for example, sensing data detected by the sensing unit 204. The sensing unit 204 detects sensing data including information related to user's chewing and/or swallowing (to be referred to as “chewing/swallowing information” hereinafter).

The sensing unit 204 is implemented as, for example, an acceleration sensor. In this case, the acceleration sensor is installed on an eating utensil that the user grips when taking a meal. Exemplary eating utensils include chopsticks, forks, and spoons. When the user chews a food, the user raises an eating utensil from a plate to pick up the food on the plate and delivers the food to the mouth, and after placing the picked up food in the mouth, the user lowers the eating utensil toward the plate again. Such motions are repeated during meal intake. As described above, raising and lowering of an eating utensil occur in conjunction with the user's chewing motion. Accordingly, acceleration information representative of an acceleration of the eating utensil represents the characteristics of the user's chewing. Accordingly, the embodiment uses, as chewing/swallowing information, acceleration information representative of an acceleration detected by an acceleration sensor installed on the eating utensil. This makes it possible to acquire chewing/swallowing information in everyday life of the user without causing too much stress to the user.

The sensing unit 204 may be implemented as an electromyographic sensor that detects electromyographic potentials. When the user chews a food, the electromyographic potentials of muscles around the jaw joint change. Accordingly, the embodiment may use, as chewing/swallowing information, electromyographic information representing the electromyographic potentials of the muscles around the jaw joint that have been detected by the electromyographic sensor. In this case, the electromyographic sensor is installed on the earpiece of eyeglasses to be worn by the user. This makes it possible to acquire chewing/swallowing information in everyday life of the user without causing too much stress to the user.

The sensing unit 204 may be implemented as a microphone. When the user chews a food, chewing sound is produced. Accordingly, the embodiment may use, as chewing/swallowing information, sound information representing sound detected by the microphone. In this case, the microphone is installed on, for example, a necklace to be worn by the user. Alternatively, the microphone may be, for example, an earphone-type microphone. If the microphone is installed on a necklace or an earphone, the installed microphone is located in proximity to the user's mouth, which allows for accurate detection of the chewing sound. This makes it possible to acquire chewing/swallowing information in everyday life of the user without causing too much stress to the user.

The sensor 200 may, for example, detect sensing data at predetermined sampling intervals, and transmit the detected sensing data at predetermined sampling intervals to the server 300 via the information terminal 100. This allows the server 300 to acquire sensing data in real time.

The server 300 includes a communications unit 301, a processor 302, and a memory 303. The communications unit 301 is implemented as a communications circuit for connecting the server 300 to the network 500. The communications unit 301 receives sensing data detected by the sensor 200 and transmitted by the information terminal 100. The communications unit 301 transmits print control information generated by the processor 302 to the food printer 400.

The processor 302 is implemented as, for example, a CPU. The processor 302 acquires chewing/swallowing information from the sensor 200 via the network 500, the chewing/swallowing information being information related to the chewing of the user when the user eats a first printed food. More specifically, the processor 302 acquires chewing/swallowing information from sensing data received by the communications unit 301. The first printed food is a food having a first hardness and created by the food printer 400 by using a material in paste form.

The processor 302 determines, based on the acquired chewing/swallowing information, the swallow cycle duration of the user, and determines, based on the first hardness and the swallow cycle duration, a second hardness for a second printed food to be created by the food printer 400. The processor 302 generates print control information for causing the food printer 400 to create the second printed food. The processor 302 transmits the generated print control information to the food printer 400 via the communications unit 301. The print control information includes information such as hardness data representing the hardness of a printed food, and three-dimensional geometry data representing the geometry of the printed food. The three-dimensional geometry data may include information such as, for example, what kind of paste is to be used where on the printed food.

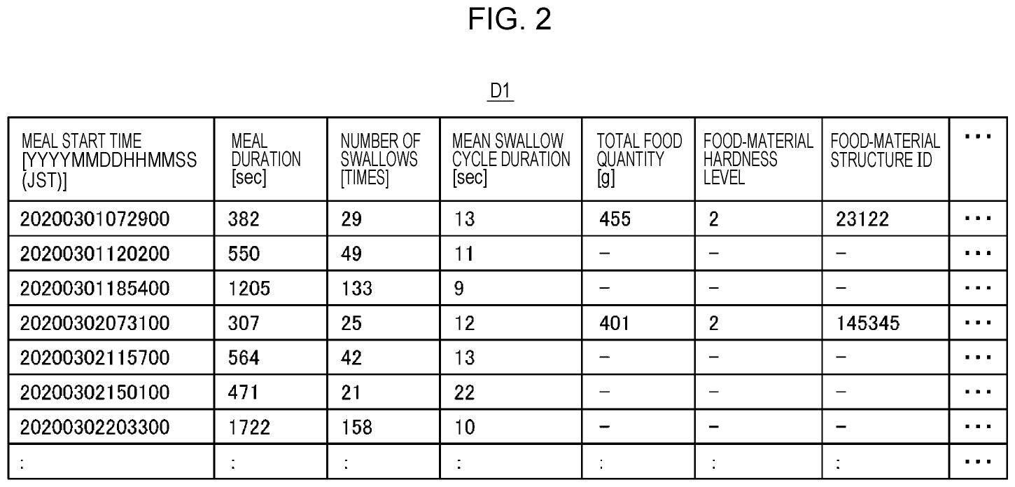

The memory 303 is implemented as a mass storage device such as a hard disk drive or a solid-state drive. The memory 303 stores a chewing database that manages user's chewing/swallowing information. FIG. 2 illustrates an exemplary data structure of a chewing/swallowing information database D1.

A single record in the chewing/swallowing information database D1 stores chewing/swallowing information associated with a single meal. A single meal corresponds to, for example, a meal such as breakfast, lunch, or dinner. The chewing/swallowing information database D1 stores, with respect to a given single user, chewing/swallowing information for each of meals such as breakfast and lunch. The example in FIG. 2 provides that the user is to eat only a printed food created by a food printer for every breakfast. Symbols “-” in the chewing/swallowing information database D1 indicate that the corresponding pieces of information have not been successfully obtained.

The chewing/swallowing information database D1 stores the following and other pieces of information in association with each other: meal start time, meal duration, the number of swallows, mean swallow cycle duration, total food quantity, food-material hardness level, and food-material structure ID. Meal start time represents the start time of a single meal. For example, for a case where the sensor 200 is implemented as an acceleration sensor, if the acceleration sensor of the processor 302 detects an acceleration waveform representative of raising or lowering of an eating utensil after such acceleration waveform has not been detected for a certain period of time, the time at which the waveform is detected is identified as the meal start time. Alternatively, the user may input a command to the information terminal 100 that signals the start of a meal, and the time at which the server 300 receives the command may be used to represent the meal start time.

Meal duration is the duration of time taken to eat a single meal. The processor 302 calculates the meal duration as the period of time from the meal start time to the meal end time. As for the meal end time, for example, when a predetermined amount of time or more elapses after a change in sensing data ceases to be observed, the timing at which a change in sensing data ceases to be observed corresponds to the meal end time. Alternatively, the user may input a command to the information terminal 100 that signals the end of a meal, and the time at which the server 300 receives the command may be used as the meal end time.

The number of swallows represents the number of times the user has swallowed food during a single meal. To determine the number of swallows, the processor 302 may analyze chewing/swallowing information acquired from the sensor 200 to identify each individual swallow cycle duration, and count how many times such a swallow cycle duration has been repeated.

Swallow cycle duration represents the period of time from when the user starts chewing a bite of food to when the user swallows the bite of food. To identify each individual swallow cycle duration, for example, if the sensor 200 is implemented as an acceleration sensor, the processor 302 may analyze acceleration information acquired from the acceleration sensor, and detect the timing of raising of an eating utensil (first timing) or the timing of lowering of an eating utensil (second timing) to thereby identify the beginning of the current swallow cycle duration. The processor 302 may then determine the time interval between the beginning of the current swallow cycle duration and the beginning of the next swallow cycle duration as representing one swallow cycle duration. Chewing is sometimes paused after a bite of food is swallowed. After a meal is finished, chewing does not occur until the next meal is started. Accordingly, if detection of the beginning of the current swallow cycle duration is not followed by detection of the beginning of the next swallow cycle duration for a predetermined period of time or more, the processor 302 may regard the moment of elapse of the predetermined period of time as representing the end of the current swallow cycle duration, and thus identify each swallow cycle duration. Alternatively, the processor 302 may regard the timing at which an eating utensil is lowered and stops moving as representing the end of the current swallow cycle duration, and thus identify each swallow cycle duration. The timing of raising or lowering of an eating utensil can be detected through, for example, pattern matching between a predefined acceleration waveform representative of raising of the eating utensil or a predefined acceleration waveform representative of lowering of the eating utensil, and acceleration information acquired from the acceleration sensor.

If the sensor 200 is implemented as an electromyographic sensor, the processor 302 may, for example, analyze electromyographic information acquired from the electromyographic sensor to detect the start timing and end timing of chewing for a bite of food, and determine the time interval between the start timing and the end timing as the swallow cycle duration. It is presumed that for a bite of food, the electromyographic potential changes in a specific pattern during the period of time from the start of chewing to the moment of swallowing. Accordingly, the processor 302 may detect, from electromyographic information, the timing of chewing initiation and the timing of swallowing with respect to a bite of food by using pattern matching or other methods, and detect the period of time between these two timings as the swallow cycle duration.

If the sensor 200 is implemented as a microphone, the processor 302 may, for example, analyze sound information acquired from the microphone to detect the timing of occurrence of chewing sound, which represents the timing of chewing initiation with respect to a bite of food, and the timing of swallowing, which represents the timing when the bite of food is swallowed, and the processor 302 may then determine the time interval between these two timings as the swallow cycle duration. For a bite of food, chewing sound is produced when chewing is initiated, and swallowing sound is produced at the timing of swallowing. Accordingly, the processor 302 may detect such chewing sound and swallowing sound from sound information by using pattern matching or other methods.

Mean swallow cycle duration is defined as the mean of the swallow cycle durations within a single meal. Mean swallow cycle duration is calculated as, for example, the meal duration divided by the number of swallows. However, this is intended to be illustrative only. Alternatively, mean swallow cycle duration may be calculated by finding the mean of swallow cycle durations detected within a single meal.

Total food quantity is defined as the total weight of food taken by the user in a single meal. The present example provides that the user is to eat a printed food for every breakfast. Since it is the server 300 that instructs that the printed food be created, the server 300 is able to determine the weight of the printed food that the user eats for every breakfast, from the weight of a paste used for creating the printed food. Accordingly, for breakfast, the processor 302 may calculate the total weight from the weight of a paste that the processor 302 has specified when generating print control information. In this regard, whether a given piece of chewing/swallowing information pertains to breakfast can be determined from the meal start time corresponding to the piece of chewing/swallowing information.

In the example in FIG. 2, the total food quantity has not been successfully identified for meals other than breakfast, and thus the Total Food Quantity cells corresponding to the chewing/swallowing information for meals other than breakfast are marked “-”. It is to be noted, however, that if the total food quantity has been successfully detected for a meal other than breakfast, the detected total food quantity is written into the chewing/swallowing information database D1. For example, when taking a meal, the user is made to capture an image of the prepared meal with a camera and have the captured image transmitted to the server 300. The processor 302 may then analyze the captured image of the prepared meal to determine the total food quantity. Alternatively, if a weight sensor is installed on the eating utensil being used, the processor 302 may determine the total food quantity by adding up the weight of each bite of food detected by the weight sensor over the entire duration of a single meal.

Food-material hardness level is a numerical value representing a graded measure of the chewing force (biting force) and swallowing force required for eating a food material. As for the food-material hardness level, for example, the classification for different classes of food materials described at the website “https://www.udfjp/about_udf/section_01.html” may be used. The lower the hardness level of a food material, the harder the food material. In the example in FIG. 2, the food-material harness level has not been successfully identified for meals other than breakfast, which is a meal for which only a printed food is to be eaten, and thus the Food-Material Hardness Level cells corresponding to the chewing/swallowing information for meals other than breakfast are marked “-”. It is to be noted, however, that if the food-material hardness level has been successfully identified through analysis of an image of a prepared meal, the identified food-material hardness level is written into the chewing/swallowing information database D1.

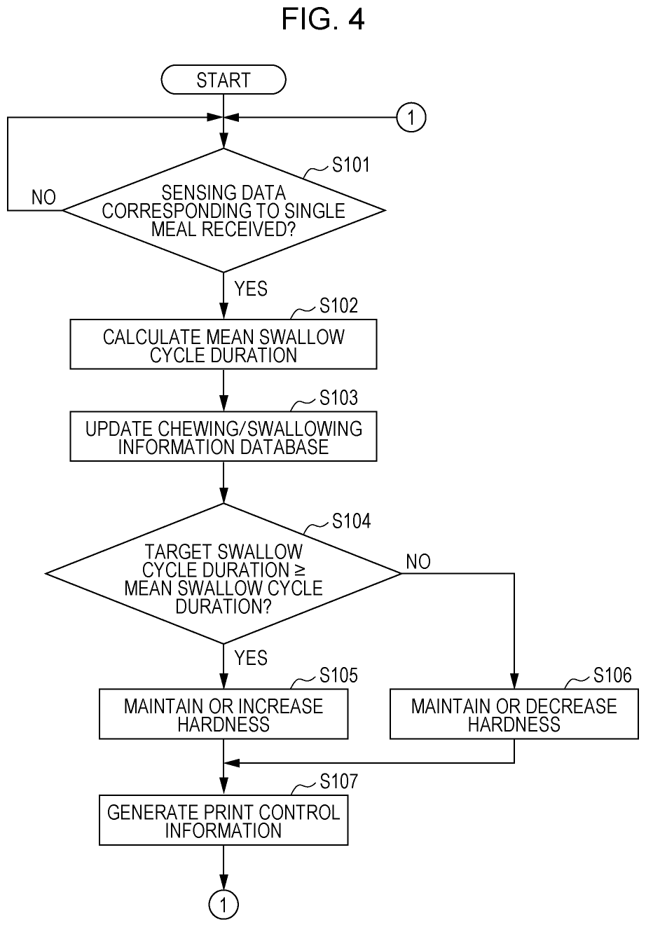

The processor 302 may determine which one of the above-mentioned classes a hardness set at step S105 or step S106 described later with reference to FIG. 4 corresponds to, and write the determined class into the corresponding Food-Material Hardness Level cell.

Food-material structure ID is an identifier of the three-dimensional geometry data of a printed food created by the food printer 400. The three-dimensional geometry data is, for example, CAD data. In the example in FIG. 2, the food-material structure ID is written only for the chewing/swallowing information corresponding to breakfast for which the printed food is eaten.

In the example in FIG. 2, the chewing/swallowing information database D1 stores chewing/swallowing information for each single meal. However, this is not intended to limit the present disclosure. For example, the chewing/swallowing information database D1 may store chewing/swallowing information for each single swallow. Alternatively, the chewing/swallowing information database D1 may store chewing/swallowing information every time a bite of food is swallowed. Although the chewing/swallowing information database D1 in FIG. 2 stores chewing/swallowing information for a given single user, the chewing/swallowing information database D1 may store chewing/swallowing information for plural users. In this case, providing the chewing/swallowing information database D1 with a user ID field makes it possible to identify which piece of chewing/swallowing information corresponds to which user.

Reference now returns to FIG. 1. The food printer 400 is a food preparation apparatus that shapes a food by dispensing a gelled food material (paste) and depositing the dispensed food material in layers.

The food printer 400 includes a communications unit 401, a memory 402, a paste dispenser 403, a controller 404, a UI unit 405, and a laser output unit 406. The communications unit 401 is implemented as a communications circuit for connecting the food printer 400 to the network 500. The communications unit 401 receives print control information from the server 300. The memory 402 is implemented as a rewritable non-volatile storage device such as a flash memory. The memory 402 stores print control information transmitted from the server 300.

The paste dispenser 403 includes plural slots, and a nozzle for dispensing a paste loaded in each slot. Each slot can be loaded with a different type of paste. Each paste is a food material packaged according to its type. The paste to be used can be replaced with respect to the paste dispenser 403. The paste dispenser 403 repeats a process of dispensing a paste while moving the nozzle in accordance with print control information. The paste is thus deposited in sequential layers to thereby shape a printed food.

The laser output unit 406 applies, in accordance with print control information, a laser beam to the paste dispensed by the paste dispenser 403. The laser output unit 406 thus heats a portion of the paste to brown a printed food or shape a printed food. The laser output unit 406 is also capable of adjusting the power of the laser beam to adjust the temperature at which to bake a printed food to thereby adjust the hardness of the printed food. The food printer 400 is capable of causing the paste dispenser 403 to discharge a paste while causing the laser output unit 406 to apply a laser beam. This makes it possible to simultaneously perform shaping and thermal cooking of the printed food.

A setting as to which slot of the paste dispenser 403 is loaded with which paste can be made by using a smartphone application installed on the information terminal 100 that communicates with the food printer. Alternatively, this setting can be made by reading, with a reader attached to each slot, a paste ID stored in an electric circuit attached to the package of a paste, and outputting the read paste ID to the controller 404 in association with the corresponding slot number.

The UI unit 405 is implemented as, for example, a touchscreen display. The UI unit 405 receives an input of a user's instruction, or displays various screens.

The controller 404 is implemented as a CPU or a dedicated electric circuit. The controller 404 creates a printed food by controlling the paste dispenser 403 and the laser output unit 406 in accordance with print control information transmitted from the server 300.

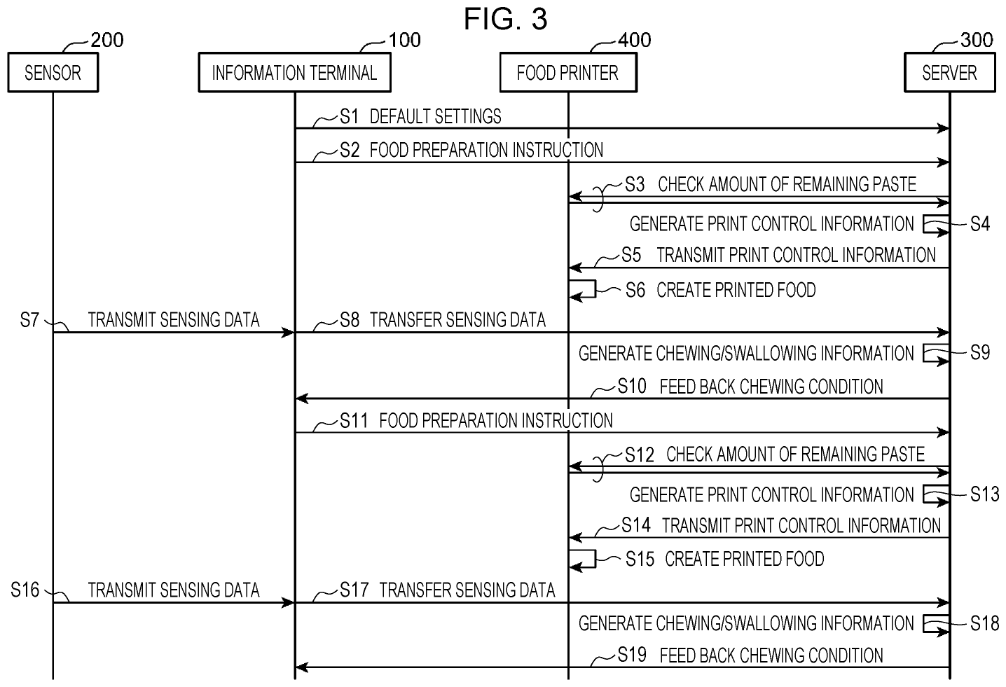

Reference is now made to processing according to the embodiment. FIG. 3 is a sequence diagram illustrating an overview of processing performed by the information system illustrated in FIG. 1.

At step S1, the information terminal 100 receives a user's input related to default settings information required for the user to receive a service from the server 300, and transmits the default settings information to the server 300. The default settings information includes, for example, a target swallow cycle duration (an example of a predetermined cycle duration), which is a target swallow cycle duration for chewing a bite of food. Since the swallow cycle duration is proportional to the number of chews, the number of chews increases with increasing swallow cycle duration. The target number of chews for chewing a bite of food is about 30. Accordingly, the target swallow cycle duration to be used may be, for example, a predefined swallow cycle duration necessary for achieving a target number of chews. The target swallow cycle duration is, for example, an amount of time such as 10 seconds, 20 seconds, or 30 seconds.

Subsequently, at step S2, the information terminal 100 receives a user's input of a food preparation instruction, which is an instruction for causing the food printer 400 to start preparation of a printed food, and transmits the instruction to the server 300.

Subsequently, at step S3, the server 300 transmits a check signal for causing the food printer 400 to check the amount of remaining paste, and receives a response from the food printer 400. In response to receiving the check signal, the food printer 400 detects, for example, the amount of paste remaining in the paste dispenser 403. If the amount of remaining paste is greater than or equal to a predetermined value, the food printer 400 transmits a response to the server 300 that indicates that creation of the printed food is possible. If the amount of remaining paste is less than the predetermined value, the food printer 400 transmits a response to the server 300 that indicates that creation of the printed food is not possible. In this case, the server 300 may transmit a message to the information terminal 100 that prompts the user to load more paste, and wait on standby until the server 300 receives a response indicating that creation of the printed food is possible.

Subsequently, at step S4, the server 300 generates print control information. Further details about the generation of print control information will be given later with reference to FIG. 4.

At step S5, the server 300 transmits the print control information to the food printer 400. Since no sensing data for a user who has eaten the printed food has been obtained at this point, the server 300 generates the print control information based on, for example, the default hardness of the printed food. The default hardness corresponds to an example of the first hardness.

At step S6, the food printer 400 creates the printed food in accordance with the received print control information. The printed food created at this time corresponds to an example of the first printed food. At step S7, the sensor 200 transmits sensing data to the information terminal 100. The sensing data includes the chewing/swallowing information of the user who has eaten the printed food created at step S6. At step S8, the information terminal 100 transfers the sensing data transmitted at step S7 to the server 300.

At step S9, the server 300 generates chewing/swallowing information associated with a single meal based on the sensing data transmitted to the server 300, and updates the chewing/swallowing information database D1 by using the chewing/swallowing information.

At step S10, the server 300 generates chewing condition data based on the chewing/swallowing information generated at step S9, and transmits the chewing condition data to the information terminal 100 to provide feedback of the chewing condition to the user. The chewing condition data includes, for example, the information illustrated in FIG. 2, such as meal duration, the number of swallows, mean swallow cycle duration, total food quantity, and food-material hardness level. The chewing condition data is displayed on the display 106 of the information terminal 100.

At step S11, the information terminal 100 transmits the food preparation instruction described above with reference to step S2 to the server 300. At step S12, the server 300 checks the amount of paste remaining in the food printer 400 in the same manner as step S3.

At step S13, the server 300 compares the mean swallow cycle duration included in the chewing/swallowing information generated at step S9 with a target mean swallow cycle duration, and based on the comparison result, the server 300 determines a hardness for the printed food, and generates print control information based on the determined hardness. Further details about this process will be given later with reference to the flowchart of FIG. 4. The hardness determined at this time corresponds to an example of the second hardness. The printed food created in accordance with the print control information generated at this time corresponds to an example of the second printed food.

Steps S14, S15, S16, S17, S18, and S19 are similar to steps S5, S6, S7, S8, S9, and S10. Thereafter, the processing from steps S11 to S19 is repeated, and the chewing and swallowing function of the user is gradually improved.

FIG. 4 is a flowchart according to the embodiment, providing a detailed illustration of processing performed by the server 300. The processor 302 of the server 300 determines whether sensing data corresponding to a single meal for a printed food has been received by the communications unit 301 (step S101). For example, as for the start timing of a single meal (meal start time), when a change is observed in the sensing data provided from the sensor 200 after no change in the sensing data has been observed for a predetermined amount of time or more, the timing of the observed change corresponds to the start timing. As for the end timing of a single meal (meal end time), for example, when a predetermined amount of time or more elapses after a change in the sensing data ceases to be observed, the timing at which a change in the sensing data ceases to be observed corresponds to the end timing of a single meal. In the example in FIG. 2, a printed food is eaten for every breakfast. Accordingly, if the start timing of a meal falls within the time of day for breakfast, the processor 302 may determine that the sensing data corresponding to a single meal acquired at step S101 represents sensing data for the printed food. Alternatively, the sensing data corresponding to a single meal acquired most recently after transmission of print control information may be determined as sensing data for the printed food. Alternatively, if an indication of the start of a meal and an indication of the end of the meal have been input by the user to the information terminal 100, a series of sensing data acquired in this case may be determined to be sensing data corresponding to a single meal.

At step S102, the processor 302 calculates the mean swallow cycle duration from the sensing data corresponding to a single meal. Since the details of how to calculate the mean swallow cycle duration have been described above, no further description in this regard will be provided herein. At step S102, in addition to calculation of the mean swallow cycle duration, values such as the meal duration, the number of swallows, and the total food quantity are also calculated, and the chewing/swallowing information illustrated in FIG. 2 is generated based on the results of these calculations.

At step S103, the processor 302 updates the chewing/swallowing information database D1 by using the chewing/swallowing information calculated at step S102.

At step S104, the processor 302 determines whether a target swallow cycle duration is greater than or equal to the mean swallow cycle duration. If the target swallow cycle duration is greater than or equal to the mean swallow cycle duration (YES at S104), the processor 302 causes the hardness of the printed food to be maintained or increased relative to the previous value. The previous value refers to the value of the hardness of the printed food last eaten by the user. The hardness represented by the previous value corresponds to an example of the first hardness. In increasing the hardness of the printed food, the processor 302 may add a predefined amount of change of hardness to the previous value to thereby increase the hardness.

If the target swallow cycle duration is less than the mean swallow cycle duration (NO at S104), the processor 302 causes the hardness of the printed food to be maintained or decreased relative to the previous value (step S106). In decreasing the hardness of the printed food, the processor 302 may subtract the amount of change mentioned above from the previous value to thereby decrease the hardness. Exemplary conceivable cases where the hardness is maintained include when the number of times that the printed food of the same hardness has been given to the user is less than a predetermined number of times.

At step S107, the processor 302 generates print control information based on the hardness that has been maintained, increased, or decreased, and returns the processing to step S101.

As the above-mentioned processing is repeated, for a user with the target swallow cycle duration greater than or equal to the mean swallow cycle duration, the hardness of the printed food is maintained or gradually increased. Accordingly, a user with decreased chewing and swallowing function is given a somewhat soft printed food at first, and then sequentially given printed foods with gradually increased hardness. This helps to efficiently improve the chewing and swallowing function of such a user.

As for a user with the target swallow cycle duration less than the mean swallow cycle duration, the hardness of the printed food is maintained or gradually decreased. Therefore, for a user with an excessively long swallow cycle duration, the swallow cycle duration is allowed to progressively converge to an appropriate value.

Detailed reference is now made to generation of print control information. According to the embodiment, the hardness of a printed food is adjusted by using one of the three variations of approaches described below. Accordingly, the print control information to be generated differs depending on which variation is used.

In the first variation, a printed food is formed as a three-dimensional structure with plural holes, and the number of these holes is increased or decreased to adjust the hardness of the printed food. A printed food becomes softer as the number of holes in the printed food increases, and harder as the number of holes decreases. Accordingly, in the first variation, the hardness of a printed food is adjusted by specifying the number of holes per unit volume of the printed food. Such adjustment of the number of holes can be made by changing three-dimensional geometry data.

Once the processor 302 of the server 300 determines the hardness of the printed food at step S105 or step S106, the processor 302 determines the number of holes per unit volume that is previously defined for achieving the hardness. The processor 302 then extracts or generates three-dimensional geometry data for creating a printed food that has a specified number of holes per unit volume.

For example, the processor 302 may correct the default three-dimensional geometry data such that the number of holes per unit volume in the default three-dimensional geometry data becomes equal to the specified number of holes per unit volume. All holes may or may not have the same diameter. One non-limiting example of the basic geometry of the default three-dimensional geometry data is a cuboid. Three-dimensional geometry data generated by the processor 302 already reflects a hardness as determined by the number of holes per unit volume. Therefore, according to the first variation, print control information may include three-dimensional geometry data generated by the processor 302, and may not include hardness data.

However, this is intended to be illustratively only. Alternatively, for example, the controller 404 of the food printer 400 may correct the default three-dimensional geometry data from hardness data. In this case, hardness data and the default three-dimensional geometry data may be included in print control information.

In the second variation, a printed food is formed as a three-dimensional structure with plural layers, and the individual layers are varied in hardness to thereby increase or decrease the hardness of the printed food. For example, a food with a hard surface and a soft interior such as rice cracker can give the user a texture sensation such that as the user crushes its hard surface with the teeth, its contents with taste mix with saliva and melt out from the inside. This induces saliva production, which helps to efficiently improve the chewing and swallowing function of the user. Accordingly, in the second variation, for example, the printed food includes a first layer having a third hardness, and a second layer having a fourth hardness lower than the third hardness. The printed food is created by stacking the first layer, the second layer, and the first layer in this order.

In this case, the processor 302 of the server 300 determines, as each of the third hardness and the fourth hardness, a predefined hardness with respect to the hardness set at step S105 or step S106. The processor 302 may then generate print control information including three-dimensional geometry data, the third hardness, and the fourth hardness. In this case, the three-dimensional geometry data may include data indicating which region corresponds to the first layer and which region corresponds to the second layer. In the second variation mentioned above, the hardness adjustment for the first and second layers may be made based on the number of holes described above with reference to the first variation. Alternatively, the hardness adjustment may be made by varying the type of paste. In this case, print control information may include information that specifies the type of paste used for the first layer and the type of paste used for the second layer.

Although a printed food has been described above as being made up of a second layer sandwiched by two first layers, a printed food may be made up of a first layer and a second layer. Further, if a printed food is made up of a second layer sandwiched by two first layers, the printed food may have a structure such that the first layer includes plural sub-layers of differing hardness, and that the second layer includes plural sub-layers of differing hardness, with the hardness of the resulting printed food decreasing gradually with increasing distance from the surface toward the center.

In the third variation, the hardness of a printed food is adjusted by specifying the temperature at which to bake the printed food. The temperature at which to bake a printed food is adjusted by adjusting the power of the laser beam to be applied. The hardness of a printed food can be changed by adjusting this temperature. In this case, the processor 302 may determine a predefined temperature required for achieving the hardness set at step S105 or S106, and incorporate temperature information representing the temperature into print control information. In this case, the print control information may include temperature information, three-dimensional geometry data, and information representing the type of the paste to be used.

Various parameters included in print control information correspond to an example of a printing condition for, if the swallow cycle duration of the user is less than a predetermined cycle duration, creating the second printed food having the second hardness greater than the first hardness.

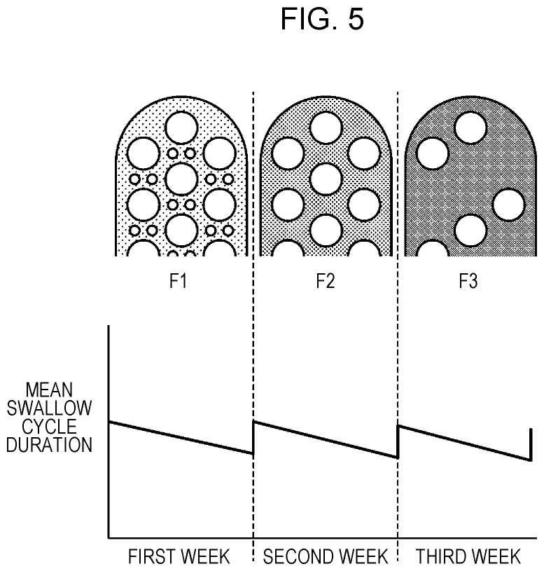

FIG. 5 illustrates the progression of mean swallow cycle duration over time. In this example, the flowchart in FIG. 4 is conducted on a weekly basis, and a printed food of the same hardness is provided to the user every morning for each week. In the first week, the user eats a printed food of a hardness F1 every morning. As the user thus gets used to the printed food of the hardness F1, the user's chewing and swallowing function gradually improves, and the mean swallow cycle duration gradually decreases.

At the beginning of the second week, it is determined whether the mean swallow cycle duration is greater than or equal to a target swallow cycle duration. At this point, the mean swallow cycle duration is not greater than the target swallow cycle duration. Accordingly, the user is given a printed food every morning that has a hardness F2, which is a hardness increased from the hardness F1 by a predetermined amount of change. Although this causes the user to increase the mean number of chews for a while to crush the printed food of the hardness F2 with the teeth, the user's chewing and swallowing function then gradually improves, which leads to progressively decreasing mean swallow cycle duration. Likewise, in the third week, the user is given a printed food every morning that has a hardness F3, which is a hardness increased from the hardness F2 by a predetermined amount of change. Although this causes the user to increase the mean number of chews for a while to crush the printed food of the hardness F3 with the teeth, the user's chewing and swallowing function then gradually improves, which leads to progressively decreasing mean swallow cycle duration. Thereafter, until the mean swallow cycle duration exceeds the target swallow cycle duration, the hardness of the printed food given to the user is gradually increased, which allows the user's chewing and swallowing function to improve progressively.

The present disclosure may take various modifications as given below.

(1) Although FIG. 1 depicts an example in which the sensor 200 transmits sensing data to the server 300 via the information terminal 100, alternatively, the sensor 200 may be connected to the network 500. In this case, sensing data may be transmitted by the sensor 200 to the server 300 without passing through the information terminal 100.

(2) The sensor 200 may be implemented as a camera. In this case, the sensor 200 is placed in a room where the user takes a meal. Generally speaking, cameras (edge terminals) have advanced processing capabilities. This means that by analyzing an image captured with such a camera, the mean swallow cycle duration can be calculated or inferred by using a neural network model. Accordingly, in this modification, the processor 202 of the sensor 200 calculates the mean swallow cycle duration by analyzing an image captured by the sensing unit 204. Chewing/swallowing information representing the calculated mean swallow cycle duration is then incorporated into sensing data, and transmitted to the server 300.

In this case, the mean swallow cycle duration is included in the chewing/swallowing information. The server 300 is thus able to determine whether the mean swallow cycle duration is greater than or equal to a target swallow cycle duration, without calculating the mean swallow cycle duration. This allows for reduced processing load on the server 300.

If a camera is used to measure chewing and swallowing through analysis, by also analyzing the lateral movements of the upper and lower jaws to measure the number of times food is chewed with the right teeth, and the number of times food is chewed with the left teeth, the user's uneven chewing can be also measured. If the difference in the number of chews between the right and left sides is greater than a predetermined value (i.e., if uneven chewing is suspected), the server 300 may register the number of chews on the left side and the number of chews on the right side into the chewing/swallowing information database D1 individually. Notification of information indicative of such uneven chewing may be provided to the user via the information terminal 100 at step S10 or S19 to allow the user to have the consciousness or motivation to improve uneven chewing (i.e., make the number of chews more even between the left and right sides). For example, the chewing balance between the right and left sides may be presented in quantified or visualized form. It is difficult for the user to notice uneven chewing on his or her own, which occurs as the jaws or masticatory muscles on the habitual chewing side become strained while the masticatory muscles on the other side relax and which can lead to misaligned jaws and consequently misalignment or distortion of the entire body. Such uneven chewing can be expected to be prevented or improved by measuring the uneven chewing with the sensor 200, and providing appropriate feedback to the user via the information terminal 100 as described above.

The condition of uneven chewing mentioned above may be measured not by using a camera but by measuring the electromyographic potential or momentum of each of the left and right masticatory muscles of the user's face. During chewing, the masticatory muscle (at least one of the masseter muscle, the temporalis muscle, the lateral pterygoid muscle, or the medial pterygoid muscle) on either the right side or left side on which the user tends to chew habitually is used more than that on the other side. Accordingly, the condition of the user's uneven chewing can be measured also by measuring the electromyographic potential or momentum of each of the left and right masticatory muscles.

According to this modification, the processor 202 may, for example, apply a predetermined image recognition process for detecting whether the user is chewing to an image captured by the sensing unit 204. In this way, the processor 202 may detect values such as the meal duration and the number of swallows within a single meal, and calculate the mean swallow cycle duration. For example, the processor 202 may detect features of the user's mouth, and keep track of the features. If the behaviors of the tracked features are representative of repeated opening and closing movements of the upper and lower jaws, the processor 202 may determine that the user is making chewing motion. The processor 202 may calculate the meal duration and the number of swallows from the detection results, and calculate the mean swallow cycle duration or other values from these calculated values.

According to this modification, the sensing unit 204 is capable of capturing an image of a prepared meal. The processor 202 is thus able to calculate the total food quantity by analyzing the image of the prepared meal. According to this modification, the processor 202 may incorporate, in addition to the mean swallow cycle duration, the following pieces of information associated with a single meal into chewing/swallowing information: the meal duration, the number of swallows, and the total food quantity.

Aspects of the present disclosure make it possible to efficiently improve chewing and swallowing function, and therefore find utility in industrial fields aimed at promoting health.

What is claimed is:

1. A method for controlling a food printer in a food-material providing system, the food printer being used to create a first printed food having a first hardness by using a material in paste form, the method comprising:
acquiring chewing/swallowing information via a network from a sensing device with which a user is equipped, wherein the chewing/swallowing information is related to chewing of the user when the user eats the first printed food;
determining based on the chewing/swallowing information, a swallow cycle duration of the user, and determining based on the first hardness and the swallow cycle duration, a second hardness for a second printed food to be created by the food printer; and
transmitting print control information to the food printer via the network, wherein the print control information is used for causing the food printer to create the second printed food having the determined second hardness.

2. The method according to claim 1,
wherein the swallow cycle duration corresponds to a period of time from when the user starts chewing a bite of the first printed food to when the user swallows the bite of the first printed food.

3. The method according to claim 1, wherein
the print control information includes a print condition for, if the swallow cycle duration of the user is less than a predetermined cycle duration, creating the second printed food having the second hardness greater than the first hardness.

4. The method according to claim 1, wherein
the sensing device includes an acceleration sensor, and
the chewing/swallowing information includes acceleration information, the acceleration information representing an acceleration detected by the acceleration sensor.

5. The method according to claim 4, wherein
the acceleration sensor is installed on one of a chopstick, a fork, or a spoon of the user, and
a beginning of the swallow cycle duration is determined by using one of a first timing determined based on the acceleration information or a second timing determined based on the acceleration information, wherein the first timing represents a timing when the user raises the one of a chopstick, a fork, or a spoon, and the second timing represents a timing when the user lowers the one of a chopstick, a fork, or a spoon.

6. The method according to claim 1, wherein
the sensing device detects an electromyographic potential, and
an end of the swallow cycle duration is determined based on the detected electromyographic potential.

7. The method according to claim 6, wherein
the sensing device is installed on eyeglasses of the user.

8. The control method according to claim 1, wherein
the sensing device detects chewing sound, and
an end of the swallow cycle duration is determined based on the detected chewing sound.

9. The method according to claim 8, wherein
the sensing device includes a microphone installed on a necklace of the user.

10. The method according to claim 8, wherein
the sensing device includes an earphone-type microphone of the user.

11. The method according to claim 1, wherein
the second printed food includes a three-dimensional structure including a number of holes, and
the second hardness is adjusted by increasing or decreasing the number of holes.

12. The method according to claim 11, wherein
the print control information specifies the number of holes per unit volume.

13. The method according to claim 1, wherein
the second printed food comprises a three-dimensional structure including a plurality of layers, wherein the plurality of layers includes a first layer with a third hardness and a second layer with a fourth hardness, and
the print control information includes a print condition for causing the third hardness to be greater than the fourth hardness.

14. The method according to claim 1, wherein
the print control information specifies a temperature at which to bake the second printed food.

15. A method for controlling a food printer in a food-material providing system, the food printer being used to create a first printed food having a first hardness by using a material in paste form, the method comprising:
acquiring chewing/swallowing information via a network from a sensing device with which a user is equipped, wherein the chewing/swallowing information represents a swallow cycle duration of the user when the user eats the first printed food;
determining based on the first hardness and the chewing/swallowing information, a second hardness for a second printed food to be created by the food printer; and
transmitting print control information to the food printer via the network, wherein the print control information being used for causing the food printer to create the second printed food having the determined second hardness.

16. The method according to claim 15, wherein
the swallow cycle duration corresponds to a period of time from when the user starts chewing a bite of the first printed food to when the user swallows the bite of the first printed food.

17. The method according to claim 15, wherein
the print control information includes a print condition for, if the swallow cycle duration of the user is less than a predetermined cycle duration, creating the second printed food having the second hardness greater than the first hardness.

18. The method according to claim 15, wherein
the sensing device includes a camera, and
a beginning and an end of the swallow cycle duration of the user are determined based on a result of image recognition performed by using an image obtained with the camera.
